(19) United States
(12) Patent Application Publication
(10) Pub. No.: US 2024/0132723 A1
(43) Pub. Date: Apr. 25, 2024

(54) MOLDED ARTICLE, MOLDING MATERIAL, JOINING STRUCTURE, METHOD FOR PRODUCING MOLDING MATERIAL, METHOD FOR PRODUCING MOLDED ARTICLE, AND METHOD FOR JOINING MOLDED ARTICLES

(71) Applicants: NISSHA CO., LTD., Kyoto-shi, Kyoto (JP); Daiho Industrial Co., Ltd., Moriguchi-city, Osaka (JP)

(72) Inventors: Tetsu NISHIKAWA, Kyoto-shi (JP); Keisuke MATSUZAKA, Moriguchi-city (JP)

(21) Appl. No.: 18/546,478

(22) PCT Filed: Feb. 1, 2022
(86) PCT No.: PCT/JP22/03749

§ 371 Date: Aug. 14, 2023

(30) Foreign Application Priority Data
Mar. 21, 2021 (JP) 2021-047166

(51) Int. Cl. C08L 97/02; A61L 15/22

(52) U.S. Cl. CPC C08L 97/02; A61L 15/225; C08L 2201/06; C08L 2203/02; C08L 2205/03

(57) ABSTRACT

Provided is a molded article that enables practical production of an unprecedented product having water disintegrability that allows disposal by flushing down a flush toilet or the like and further having high aesthetics. A molded article containing pulp, carboxymethyl cellulose salt, and starch, with the ratio of the pulp, the carboxymethyl cellulose salt, and the starch satisfying pulp:carboxymethyl cellulose salt:starch=50 to 80% by mass:15 to 40% by mass:5 to 10% by mass, wherein the molded article is not for an applicator for intravaginal insertion.

### TECHNICAL FILED

The present invention relates to a molded article, a molding material, a joining structure, a method for producing a molding material, a method for producing a molded article, and a method for joining molded articles.

### BACKGROUND ART

Pulp injection molding with a molding material containing pulp and a starch-based binder as main components has been previously proposed (e.g., Patent Literatures 1, 2).

In this context, Patent Literature 2 discloses a technique of imparting good water dispersibility (water disintegrability) to a biodegradable molded article, for example, to make the molded article disposable by flushing down a flush toilet or the like in using it as a disposal article for use in toilets.

### CITATION LIST

### Patent Literature

Patent Literature 1: Japanese Patent No. 2916136
Patent Literature 2: Japanese Patent No. 6697874

### SUMMARY OF INVENTION

### Technical Problem

The molded article according to Patent Literature 2 may suffer from surface roughening such as generation of cracks in the surface, and smoother surfaces and the resulting improved surface esthetics are demanded for molded articles.

The present invention has been made in view of the circumstances as described, and an object of the present invention is to provide a molded article, a molding material, a joining structure, a method for producing a molding material, a method for producing a molded article, and a method for jointing molded articles, each of which enables practical production of an unprecedented product having water disintegrability that allows disposal by flushing down a flush toilet or the like and further having high aesthetics.

### Solution to Problem

The summary of the present invention will be described with reference to attached drawings.

A molded article containing pulp, carboxymethyl cellulose salt, and starch, with the ratio of the pulp, the carboxymethyl cellulose salt, and the starch satisfying pulp:carboxymethyl cellulose salt:starch=50 to 80% by mass:15 to 40% by mass:5 to 10% by mass, wherein the molded article is not for an applicator for intravaginal insertion.

The molded article according to claim 1, wherein the pulp is mixed pulp of softwood pulp and hardwood pulp.

The molded article according to claim 2, with the mixing ratio by mass of the softwood pulp to the hardwood pulp being 30:70 to 70:30.

The molded article according to claim 1, wherein the pulp is hardwood pulp.

A molding material containing pulp, carboxymethyl cellulose salt, starch, and water for use in injection molding or compression molding, with the ratio of the pulp, the carboxymethyl cellulose salt, and the starch satisfying pulp:carboxymethyl cellulose salt:starch=50 to 80% by mass:15 to 40% by mass:5 to 10% by mass, and the moisture content being 20 to 65% by mass.

The molding material according to claim 5, wherein the pulp is mixed pulp of softwood pulp and hardwood pulp.

The molding material according to claim 6, with the mixing ratio by mass of the softwood pulp to the hardwood pulp being 30:70 to 70:30.

The molding material according to claim 5, wherein the pulp is hardwood pulp.

The molding material according to any one of claims 5 to 8, wherein 0.3 to 2.0 parts by mass of a non-alkali metal salt of long-chain fatty acid has been added to 100 parts by mass of a mixture of the pulp, the carboxymethyl cellulose salt, and the starch.

A joining structure formed by joining pieces by engaging and pressurizing the pieces with the pieces moistened, and solidifying the pieces by drying, wherein the pieces are formed by molding the molding material according to any one of claims 5 to 9, and not for an applicator for intravaginal insertion.

The joining structure according to claim 10, wherein the joining by engaging and pressurizing is performed by applying 10 g/m 2 or more of water to an engaged portion and retaining an applied pressure equal to or higher than 10 kPa for 10 sec or longer.

A method for producing a molding material containing pulp, carboxymethyl cellulose salt, starch, and water for use in injection molding or compression molding, wherein the pulp, the carboxymethyl cellulose salt, the starch, and the water are mixed and kneaded.

The method for producing a molding material according to claim 12, with the ratio of the pulp, the carboxymethyl cellulose salt, and the starch satisfying pulp:carboxymethyl cellulose salt:starch=50 to 80% by mass:15 to 40% by mass:5 to 10% by mass, wherein a mixture of the pulp, the carboxymethyl cellulose salt, and the starch is kneaded with addition of water to 100 parts by mass of the mixture to reach a moisture content of 20 to 65% by mass.

The method for producing a molding material according to claim 12 or 13, wherein the pulp is mixed pulp of softwood pulp and hardwood pulp.

The method for producing a molding material according to claim 14, with the mixing ratio by mass of the softwood pulp to the hardwood pulp being 30:70 to 70:30.

The method for producing a molding material according to claim 12 or 13, wherein the pulp is hardwood pulp.

The method for producing a molding material according to any one of claims 12 to 16, wherein a product obtained by mixing the pulp, the carboxymethyl cellulose salt, the starch, and the water is kneaded with addition of a non-alkali metal salt of long-chain fatty acid.

The method for producing a molding material according to claim 17, with the loading of the non-alkali metal salt of long-chain fatty acid being 0.3 to 2.0 parts by mass to 100 parts by mass of a mixture of the pulp, the carboxymethyl cellulose salt, and the starch.

The method for producing a molding material according to any one of claims 12 to 18, wherein the kneading is performed at 40° C. to 90° C.

A method for producing a molded article containing pulp, carboxymethyl cellulose salt, and starch, wherein injection molding or compression molding is performed with use of the molding material according to any one of claims 5 to 9 under the following conditions:
injection speed: 10 to 30 mm/s;
injection pressure: 80 to 140 MPa; and
mold temperature: 150 to 180° C.

A method for joining molded articles each containing pulp, carboxymethyl cellulose salt, and starch, wherein the molded articles are each not for an applicator for intravaginal insertion, the method including:
an application step of applying water to facing surfaces for joining the molded articles together;
an engaging step of engaging the facing surfaces with each other; and
a drying step of drying the resulting engaged portion.

The method for joining molded articles according to claim 21, wherein the pulp is mixed pulp of softwood pulp and hardwood pulp.

The method for joining molded articles according to claim 22, with the mixing ratio by mass of the softwood pulp to the hardwood pulp being 30:70 to 70:30.

The method for joining molded articles according to claim 21, wherein the pulp is hardwood pulp.

The method for joining molded articles according to any one of claims 21 to 24, with the ratio of the pulp, the carboxymethyl cellulose salt, and the starch satisfying pulp:carboxymethyl cellulose salt:starch=50 to 80% by mass:15 to 40% by mass:5 to 10% by mass in each of the molded articles.

The method for joining molded articles according to any one of claims 21 to 25, wherein the application step is performed under the following condition:
water application rate: 10 g/m 2 or more.

The method for joining molded articles according to any one of claims 21 to 26, wherein the engaging step is performed under the following conditions:
applied pressure: 10 kPa or higher; and
pressurization retention time: 10 sec or longer.

The method for joining molded articles according to any one of claims 21 to 27, wherein the drying step is performed under the following conditions:
temperature: 50° C. or higher; and
drying time: 2 minutes or longer.

### Advantageous Effects of Invention

Configured as described above, the molded article, molding material, joining structure, method for producing a molding material, method for producing a molded article, and method for joining molded articles according to the present invention enable practical production of an unprecedented product having water disintegrability that allows disposal by flushing down a flush toilet or the like and further having high aesthetics.

### BRIEF DESCRIPTION OF DRAWINGS

FIG. 1 is a photograph showing an example of a molded article with generation of a crack.

FIG. 2 is a photograph showing an example of a molded article without generation of a crack.

FIG. 3(a) shows half parts of a molded article in Experiment 3, and FIG. 3(b) shows a cylinder obtained by joining the half parts.

FIG. 4 shows a fixing jig used in Experiment 3.

### DESCRIPTION OF EMBODIMENTS

Modes of implementation of the present invention that are thought to be preferable will be briefly described by showing the actions of the present invention with reference to the drawings.

The molded article according to the present invention, which contains a specific amount of carboxymethyl cellulose salt (CMC), has excellent water disintegrability, thus being applicable as a disposable article or the like that can be disposed, for example, by flushing down a flush toilet or the like.

In addition, inclusion not only of CMC but also of a specific amount of starch makes a surface smoother than those in the case without starch and prevents the generation of cracks, thus enabling practical production of a molded article having high aesthetics.

Furthermore, in joining two molded articles together, for example, inclusion of CMC and starch allows the two to be joined and integrated together through a process that water is applied to facing surfaces that serve as joining surfaces, making part of them gelatinous, the facing surfaces are engaged with each other, and the resulting engaged portion is dried. Accordingly, even in the case of a molded article having a complex shape, for example, including what is called undercut structure (a molded article with a dedicated shape, patterns, or the like for higher aesthetics), for example, half parts of the shape that have been formed in a manner that the generation of the undercut is avoided by molding with normal molds without use of a special mold including a slide core, an inclined core, or the like can be joined and integrated together by the aforementioned method; thus, no special mold is needed.

Therefore, the present invention enables practical production of an unprecedented biodegradable molded article that is disposable by flushing down a flush toilet or the like and at the same time has high aesthetics.

### EXAMPLES

A specific embodiment of the present invention will be described with reference to the drawings.

The present embodiment is a molded article containing pulp, carboxymethyl cellulose salt, and starch, with the ratio of the pulp, the carboxymethyl cellulose salt, and the starch satisfying pulp:carboxymethyl cellulose salt:starch=50 to 80% by mass:15 to 40% by mass:5 to 10% by mass, wherein the molded article is not for an applicator for intravaginal insertion.

Examples of the molded article of the present embodiment include packaging buffers that are water-disintegrable after use (e.g., sheets for packaging fragile articles such as glass bottles); container bodies for dry foods (e.g., storage bags and storage containers for storing grains or the like); tools for washing toilets, containers for urinalysis, and articles in lavatories such as sanitary waste receptacles and disposal potties; containers for storing fertilizers, plant seeds, or the like, wherein the tools, containers, and articles are disposable by dispersing and flowing away after use; and festival articles such as floating lanterns and floating dolls.

The molding material that is used for forming the molded article is one obtained by kneading a mixture obtained by mixing pulp, carboxymethyl cellulose salt, starch, and water with addition of a non-alkali metal salt of long-chain fatty acid, as necessary, and forming the resultant into a predetermined shape (e.g., pellets or tablets).

Natural fiber can be used as the pulp. Examples thereof include wood pulp such as papermaking pulp, dissolving pulp, mercerized pulp, and fluff pulp; non-wood pulp obtained from cotton, linters, flax, Manila hemp, sisal hemp, bagasse, kenaf, straw, or the like; waste paper pulp obtained from waste paper; and plant crushed products such as chaff and wood flour. In addition, lyocell fiber obtained by purifying cellulose in natural fiber and rayon fiber obtained by regenerating natural cellulose can be each used as one of the natural fiber.

In the present embodiment, wood pulp is used. Specifically, hardwood pulp or softwood pulp, or a product obtained by mixing hardwood pulp and softwood pulp at a specific ratio is used. Inclusion of more hardwood pulp gives better water disintegrability. In the present embodiment, the ratio in mixing hardwood pulp and softwood pulp is set to 30:70 to 70:30. The ratio is preferably 50:50.

The hardwood pulp to be used is one having a fiber length of around 0.9 mm and a fiber diameter of about 20 μm. The softwood pulp to be used is one having a fiber length of around 2 mm and a fiber diameter of about 40 μm.

The carboxymethyl cellulose salt to be used is one having a degree of etherification of 0.5 to 1.0, preferably of 0.60 to 1.00, more preferably of 0.65 to 0.75. Carboxymethyl cellulose salt having a degree of etherification lower than 0.5 gives low flowability in filling a mold with the molding material and makes the molded article brittle, thus being unpreferable, and carboxymethyl cellulose salt having a degree of etherification over 1.0 gives high moisture retention and causes the molding material filling a mold to require much time to solidify by drying, leading to lower productivity for the molded article.

For the carboxymethyl cellulose salt, the alkali metal salts, calcium salt, magnesium salt, ammonium salt, and other salts are known to be water soluble, and the alkali metal salts such as the sodium salt are preferred because good water disintegrability is imparted to the molded article, whereas the calcium salt, magnesium salt, and ammonium salt impart poor water disintegrability to the molded article. Accordingly, carboxymethyl cellulose sodium salt is employed in the present embodiment.

In the present embodiment, 0.3 to 2.0 parts by mass of a non-alkali metal salt of long-chain fatty acid has been added to 100 parts by mass of a mixture of pulp, carboxymethyl cellulose salt, and starch with the ratio of the pulp, the carboxymethyl cellulose salt, and the starch satisfying pulp:carboxymethyl cellulose salt:starch=50 to 80% by mass:15 to 40% by mass:5 to 10% by mass.

If less than 50% by mass, the formulation ratio of pulp in the molding material results in lower surface smoothness or strength in the molded article, thus being unpreferable. If over 80% by mass, the formulation ratio does not allow molding. The formulation ratio is preferably 50 to 70% by mass.

If less than 15% by mass, the formulation ratio of carboxymethyl cellulose salt results in deteriorated water disintegrability, thus being unpreferable. If over 40% by mass, the formulation ratio results in lower surface smoothness or strength in the molded article, thus being unpreferable. The formulation ratio is preferably 20 to 35% by mass.

If less than 5% by mass, the formulation ratio of starch results in deteriorated moldability and lowered aesthetics, too, thus being unpreferable. If over 10% by mass, the formulation ratio results in deteriorated water disintegrability, thus being unpreferable.

For the molding material of the present embodiment, 100 parts by mass of a mixture of the natural fiber, the carboxymethyl cellulose salt, and the starch is kneaded with addition of 50 to 190 parts by mass of water to the mixture to reach a moisture content of 20 to 65% by mass. If less than 50 parts by mass, the loading of water causes increased loads in kneading to inhibit homogeneous kneading, and, in forming the kneaded molding material into pellets or tablets, difficulty in retention of the shape. If over 190 parts by mass, the loading of water causes significantly low viscosity to give a muddy kneaded product, which is difficult to mold and dry, thus being unpreferable.

For example, natural fiber, carboxymethyl cellulose salt, starch, and water are sequentially weighed in a plastic beaker having a capacity of approximately 1 liter in such a manner that the total amount reaches 55 to 60 g, and the mixture is then stirred, and plasticized and kneaded to give the molding material.

The non-alkali metal salt of long-chain fatty acid in the present embodiment, having a nonpolar moiety derived from the fatty acid chain moiety and a polar moiety derived from the non-alkali metal moiety, is insoluble in water and has water repellency and surfactant functions, and has lubricity in the state of any of melt and powder.

Accordingly, the non-alkali metal salt of long-chain fatty acid can act as an internal lubricant in kneading the molding material to prevent the molding material from sticking onto a container wall or the like, improving the workability. In addition, the non-alkali metal salt of long-chain fatty acid acts as an external lubricant in a mold and has effects of inhibiting the molding material from sticking onto mold wall surfaces when a skin layer is formed on the surface of the molding material through drying, facilitating the formation of flow channels through which water is vaporized and removed from the molding material, and lowering the frictional resistance between the molded article and the mold wall surfaces to allow the molded article to be released without cracking.

Examples of the non-alkali metal salt of long-chain fatty acid include, but are not limited to, calcium stearate, magnesium stearate, zinc stearate, calcium laurate, magnesium laurate, zinc laurate, aluminum laurate, strontium laurate, aluminum stearate, and strontium stearate. One of these may be used alone, and two or more of them may be mixed for use.

It is preferable that 0.3 to 2.0 parts by mass of the non-alkali metal salt of long-chain fatty acid be added to 100 parts by mass of the total of pulp, carboxymethyl cellulose salt, and starch. If less than 0.3 parts by mass, the loading of the non-alkali metal salt of long-chain fatty acid results in lowering of the lubricant effect and, in the step of kneading the molding material, lowering of the function of preventing sticking, which causes a tendency of sticking onto wall surfaces or the like of a molding apparatus, and gives rise to a need for a longer time for evacuation of water vapor in the molding step. Moreover, in releasing the molded article at a small draft angle, such a loading of the non-alkali metal salt of long-chain fatty acid makes it difficult to smoothly release and may cause cracking in the molded body, thus being unpreferable.

If over 2.0 parts by mass, the loading of the non-alkali metal salt of long-chain fatty acid gives an excessively high lubricant effect in the step of kneading the molding material to reduce the frictional resistance to the wall surfaces of a kneader, thus making it difficult to uniformly knead in a short time. Moreover, such a loading of the non-alkali metal salt of long-chain fatty acid causes lowering of the strength of joining portions when the confluence of the molding material occurs inside a mold in molding, thus being unpreferable.

To the molding material obtained in the described manner, an antimicrobial agent to prevent generation of mold or a polyhydric alcohol such as glycerol to impart flexibility can be added, as necessary.

The moisture content (the proportion of moisture to the whole material) of the molded material (including the case of pellets or tablets) after kneading is, as described above, 20% by mass to 65% by mass, preferably 25% by mass to 50% by mass, and more preferably 30% by mass to 40% by mass. If less than 20% by mass, the moisture content results in significant lowering of the fluidity of the molding material to lead to difficulty in molding, thus being unpreferable. If over 65% by mass, the moisture content similarly leads to difficulty in molding because of low viscosity, and gives rise to need of long time for evacuation and release of water vapor and prolonged molding time to lower the production efficiency in injection molding, thus being unpreferable.

A mold for injection molding or a mold for compression molding is heated and filled with the molding material, which is solidified by drying through vaporization and removal of the water added, and the resultant is taken out; in this way, the molded article can be produced.

In forming a molded article having a complex shape including undercut structure by molding, it is typically needed to use a special mold including a slide core, an inclined core, or the like.

In this regard, the molding material of the present embodiment, which contains specific amounts of carboxymethyl cellulose salt and starch, allows, for example, in the case of joining two molded articles (pieces) together, the two to be joined and integrated together through a process that water is applied to facing surfaces that serve as joining surfaces to make them gelatinous, the facing surfaces are engaged with each other, and the resulting engaged portion is dried.

Simply speaking, normal molds are enough for achieving the purpose even without use of any special mold. For example, the purpose can be achieved in such a manner that half parts of a shape are formed in a manner that the generation of undercut is avoided by molding with normal molds, and the half parts are joined and integrated together by the aforementioned method.

Specifically, water is applied to facing surfaces of molded articles to make the facing surfaces gelatinous, and the facing surfaces are engaged with each other and pressurized to closely adhere them at room temperature for a specific time, and naturally dried to join and integrate the two molded articles together.

More specifically speaking, it is preferable to set the application rate of water to 10 g/m² or more and less than 1,000 g/m². If less than 10 g/m², the application rate of water does not allow the progress of gelatinization of the joining surfaces to result in lower joining strength, thus being unpreferable. If 1,000 g/m² or more, the application rate of water may cause deformation of the shapes of the molded articles and gives rise to need of excessive temperature and time for drying, thus being unpreferable. It is preferable to employ pressurizing conditions with an applied pressure equal to or higher than 10 kPa and a pressurization retention time equal to or longer than 10 sec. Pressurizing conditions with an applied pressure lower than 10 kPa or a pressurization retention time shorter than 10 sec do not allow the progress of gelatinization of the joining surfaces to result in lower joining strength, thus being unpreferable. It is preferable to employ drying conditions with a temperature equal to or higher than 50° C. and a drying time equal to or longer than 2 minutes. Drying conditions with a drying temperature lower than 50° C. or a drying time shorter than 2 minutes do not allow the gelatinized material to solidify by drying to result in lower joining strength, thus being unpreferable.

Through the process, even molded articles having a complex shape including undercut structure can be efficiently produced with higher complexity and higher cost for molds prevented, and molded articles that are disposal by flushing down a flush toilet or the like and at the same time have high aesthetics can be obtained.

The present embodiment, configured as described above, contains the specific amount of carboxymethyl cellulose salt (CMC), and as a result has excellent water disintegrability, being applicable to disposal articles that can be disposed, for example, by flushing down a flush toilet or the like. For example, the water dispersibility can be set to one such that a molded flat plate of 10 mm in length, 10 mm in width, and 1 mm in thickness put in 300 mL of water and stirred at 650 rpm begins to disperse within 10 minutes and separates into fragments of 50 mm 2 or smaller in area within 15 minutes.

In addition, inclusion not only of CMC but also of the specific amount of starch gives surface smoothness better than those in the case without starch, thus enabling practical production of a molded article having high aesthetics.

### EXPERIMENT EXAMPLES

Experiment examples to support the aforementioned effects will be described.

### Experiment 1

Experiment 1 is an experiment to check whether starch influences water disintegrability, whether starch causes generation of cracks, and others.

Comparative Example 1, the samples of which contained pulp and CMC, and Examples 1 and 2, the samples of each of which contained pulp, CMC, and starch, were compared. This pulp consisted only of softwood pulp, not containing hardwood pulp.

To obtain samples of Comparative Example 1 and Examples 1 and 2, molding materials were kneaded under conditions shown below, and molding was performed with the resulting molding materials under conditions shown below (five samples were produced for each example, and each sample was checked for the surface roughness and the presence or absence of a crack).

Conditions for Kneading

Kneading was performed by using a Labo Plastomill Model 15-50 manufactured by Toyo Seiki Seisaku-sho, Ltd. at a preset temperature of 70° C. for 2 minutes, and, immediately after kneading, molding tablets of 50 mm in diameter were formed. Temperatures of 40 to 90° C. are effective in plasticizing and kneading a molding material, and kneading temperatures lower than 40° C. result in insufficient gelatinization of starch, making it difficult to obtain a uniform molding material, and temperatures over 90° C. cause the beginning of drying of a material during kneading, thus being unpreferable. Kneading is performed in such a manner that the moisture content of the aforementioned molding material (including the case of pellets or tablets) reaches 20% by mass to 65% by mass.

Conditions for Injection Molding

The molding apparatus used was a NEX280III injection molding machine manufactured by NISSEI PLASTIC INDUSTRIAL CO., LTD., and the mold used was one including an evacuation vent and evacuation means to release and evacuate water vapor generated during molding outside the mold. In view of the possibility of the flow-in of a molding material into the evacuation vent when the inside of a cavity was filled with molding tablets and the pressure in the cavity reached 30 kgf/mm² or higher, an outlet port to release an excessive portion of a material was provided in a parting surface of the mold as a final position to be filled with a molding material, and the mold temperature was set to 150 to 180° C. The molding tablets were solid at normal temperature, immediately softened by heat and pressure in the mold to be given low viscosity, and successfully molded at a pressure equal to or lower than 5 kgf/mm² to the maximum gauge pressure of 210 kgf/mm².

The injection speed was set to 10 to 30 mm/s, and the injection pressure was set to 80 to 140 MPa. An injection speed lower than 10 mm/s or an injection pressure lower than 80 MPa may cause filling failure or shaping failure, thus being unpreferable. An injection speed over 30 mm/s or an injection pressure over 140 MPa causes generation of larger burrs in parting lines and brushing lines of the mold and may deteriorate the operation efficiency or damage the mold, thus being unpreferable.

Evaluation was carried out as follows.

Surface roughness and cracks

Evaluated by raters through visual observation.

Water disintegrability

Evaluated with time until a molded article of 0.4 mm to 1 mm in thickness was determined (sensory evaluation) to have dissolved by hand stirring.

Table 1 shows results of Experiment 1. In Table 1, each field in the row “Formulation ratio” shows a formulation ratio of pulp (Pulp), carboxymethyl cellulose salt (CMC), and starch (Starch).

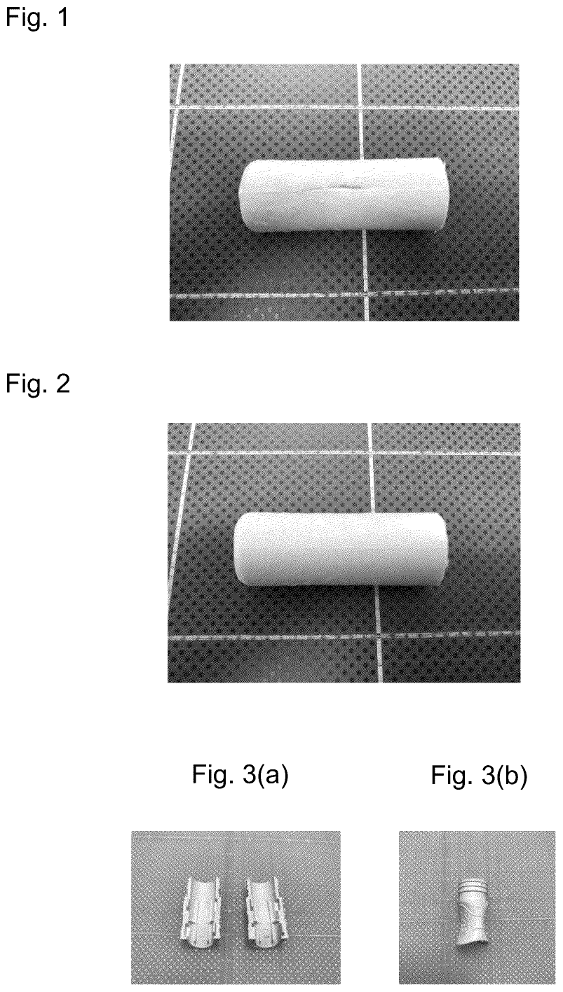

As demonstrated in Table 1, the samples of Comparative Example 1 were all poor in surface roughness, and tended to generate a crack (see FIG. 1). By contrast, the samples of Examples 1 and 2 were confirmed to be good in surface roughness, and less tend to generate a crack (see FIG. 2). Even though the starch contained in the samples of Examples 1 and 2 resulted in slightly lower water disintegrability than the samples of Comparative Example 1, the degree of lowering has been confirmed not to be problematic in disposal by flushing.

TABLE 1

| | Comparative | | |
|---|---|---|---|
| | Example 1 | Example 1 | Example 2 |
| Formulation ratio | 60/40/0 | 60/35/5 | 60/30/10 |
| (Pulp/CMC/Starch) | | | |
| Surface roughness | Poor | Good | Good |
| Crack | 1 pcs: Good | 4 pcs: Good | 5 pcs: Good |
| | 4 pcs: Crack | 1 pc: Crack | |
| Water disintegrability | 7 min | 10 min | 15 min |

### Experiment 2

Experiment 2 is an experiment to examine different formulation ratios for improved water disintegrability.

Different formulation ratios of pulp, carboxymethyl cellulose salt, and starch were compared. In addition, different formulation ratios of hardwood pulp (LBKP) and softwood pulp (NBKP) were compared.

Conditions for kneading and molding for Comparative Examples 2 and 3 and Examples 1 to 7 were the same as those in Experiment 1. Five samples were produced and evaluated for each example.

Evaluation was carried out as follows.

Moldability

Relative evaluation was carried out on the basis of Example 2, as a reference, in Table 2 (a case with the number of cracks being larger than that for Example 2 was rated as “Poor,” and a comparable case as “Good”).

Water Disintegrability

(Test method): a 500-mL beaker containing 300 mL of water (temperature of water: 20±5° C.) was placed on a magnetic stirrer, and the rotational frequency of the rotor was adjusted to 600±10 rotations/min. Thereinto, a cylindrical test piece of approximately 30 mm in height and 0.5 to 1.1 mm in thickness was put, and evaluated on a residue after stirring for 1 hour.

(Evaluation method): relative evaluation was carried out on the number and size of masses of 1 mm or larger on the basis of Example 2 as a reference (a case with the number and the size being larger than those for Example 2 was rated as “Poor,” a case with either one of the number and the size being larger than that for Example 2 as “Fair,” a case with the number and the size being comparable to those for Example 2 as “Good,” and a case with the number and the size being smaller than those for Example 2 as “Excellent”).

Table 2 shows the experimental results.

TABLE 2

| | | | | | | Water |
|---|---|---|---|---|---|---|
| | LBKP | NBKP | CMC | Starch | Moldability | disintegrability |
| Comparative | 70% | 10% | 13% | 7% | Poor (unable to mold) | — |
| Example 2 | | | | | | |
| Comparative | 10% | 70% | 13% | 7% | Poor (unable to mold) | — |
| Example 3 | | | | | | |
| Example 1 | — | 60% | 35% | 5% | Good | Excellent |
| Example 2 | — | 60% | 30% | 10% | Good (reference) | Good (reference) |
| Example 3 | 34% | 34% | 24% | 7% | Good | Good |
| Example 4 | 70% | — | 23% | 7% | Good | Excellent |
| Example 5 | 58% | — | 35% | 7% | Good | Excellent |
| Example 6 | — | 70% | 23% | 7% | Good | Fair |
| Example 7 | — | 58% | 35% | 7% | Good | Good |

As demonstrated in Table 2, the present Examples 1 to 7 gave good moldability and water disintegrability. For Comparative Examples 2 and 3, by contrast, the excessively small amounts of CMC gave rise to deterioration of moldability, eventually making molding impossible. In addition, it was confirmed that large amounts of CMC (small amounts of CMC give deteriorated water disintegrability if LBKP is absent), high ratios of LBKP, and exclusive use of LBKP result in good water disintegrability.

It was confirmed that the lowering of water disintegrability due to reduction in the amount of CMC, which is expensive, can be prevented by mixing with LBKP.

### Experiment 3

Experiment 3 is an experiment to examine the degree of adhesion in joining two molded articles with water.

Two identically shaped (semicylindrical) half parts of a molded article formed with the formulation of Example 3 in Experiment 2 above (see FIG. 3(a)) were positioned to face each other, and joined and integrated into a cylinder (see 3(b)) according to an experimental procedure in Table 3, and the degree of adhesion was then checked. Table 4 shows the experimental results.

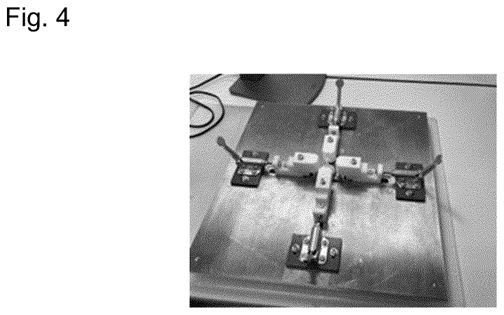

TABLE 3
Experimental
procedure

| | |
|---|---|
| (1) | Two semicylindrical molded articles are prepared (peripheral burrs are removed in advance). |
| (2) | Tap water is applied to edge surfaces of the molded articles that serve as adhesion surfaces with the |
| | tip of a syringe. The application rate was 100 g/m² or more (1,000 g/m² or less). |
| (3) | After attaching the adhesion surfaces of the molded articles to each other, the cylindrical molded |
| | article is kept under retained pressure for a specific time with a special fixing jig (see FIG. 4) to hold |
| | the outer peripheral surface of the cylindrical molded article at a specific pressure (10 kPa or higher) |
| | from the four directions. |
| (4) | After completion of pressure retention, the cylindrical molded article is removed from the jig, and |
| | placed in an oven at a specific temperature for heating. |
| (5) | After heating for the specific time, the cylindrical molded article is taken out. |
| (6) | Whether the adhesion surfaces are peeled apart is checked by fingers. |

The application rate of 100 g/m² or more is such a rate that the whole of the adhesion surfaces is wetted. Application rates lower than that do not allow the whole surface to be wetted. Application rates over 1,000 g/m² do not allow retention of the cylindrical shape because of the excessive moisture.

TABLE 4

| | Pressure | | Drying | Drying | | |
|---|---|---|---|---|---|---|
| | retention | | temperature | time | Rating on | |
| No. | time | | (° C.) | (min) | adhesion | Remarks |
| 1 | 1 | min | 50 | 5 | Poor | 6 mm of peeling-apart in one side of patterned |
| | | | | | | part |
| 2 | 1 | min | 50 | 6 | Good | |
| 3 | 10 | s | 50 | 6 | Poor | 11 mm of peeling-apart in one side of patterned |
| | | | | | | part |
| 4 | 10 | s | 80 | 5 | Good | |
| 5 | 1 | min | 80 | 3 | Fair | With gap but without peeling-apart |
| 6 | 1 | min | 80 | 2 | Poor | 5 mm of peeling-apart in one side of patterned |
| | | | | | | part |
| 7 | 1 | min | 80 | 4 | Good | |
| 8 | 10 | s | 120 | 5 | Good | |
| 9 | 1 | min | 120 | 3 | Good | |
| 10 | 1 | min | 120 | 2 | Good | |
| 11 | 1 | min | 120 | 1 | Poor | 9 mm of peeling-apart in one side of patterned |
| | | | | | | part |

The rating on adhesion was based on adhesion strength, and a case with easy peeling-apart by fingers was rated as “Poor,” a case without peeling-apart by fingers as “Good,” and a case with a gap being present between the adhesion surfaces but not allowing peeling-apart by fingers as “Fair.” The adhesion strengths for “Good” and “Fair” were confirmed to be 4,000 to 5,000 kPa.

Results of Experiment 3 confirmed that pressure retention times equal to or longer than 10 s allow adhesion. In addition, pressure retention times of about 5 s were confirmed to result in poor adhesion in the patterned part side.

Regarding drying conditions, it was confirmed that drying for 6 minutes or longer at 50° C., for 4 minutes or longer at 80° C., and for 2 minutes or longer at 120° C. allow adhesion without peeling-apart.

1. A molded article comprising pulp, carboxymethyl cellulose salt, and starch, with a ratio of the pulp, the carboxymethyl cellulose salt, and the starch satisfying pulp:carboxymethyl cellulose salt:starch=50 to 80% by mass:15 to 40% by mass:5 to 10% by mass, wherein the molded article is not for an applicator for intravaginal insertion.

2. The molded article according to claim 1, wherein the pulp is mixed pulp of softwood pulp and hardwood pulp.

3. The molded article according to claim 2, with a mixing ratio by mass of the softwood pulp to the hardwood pulp being 30:70 to 70:30.

4. The molded article according to claim 1, wherein the pulp is hardwood pulp.

5. A molding material comprising pulp, carboxymethyl cellulose salt, starch, and water for use in injection molding or compression molding, with a ratio of the pulp, the carboxymethyl cellulose salt, and the starch satisfying pulp:carboxymethyl cellulose salt:starch=50 to 80% by mass:15 to 40% by mass:5 to 10% by mass, and a moisture content being 20 to 65% by mass.

6. The molding material according to claim 5, wherein the pulp is mixed pulp of softwood pulp and hardwood pulp.

7. The molding material according to claim 6, with a mixing ratio by mass of the softwood pulp to the hardwood pulp being 30:70 to 70:30.

8. The molding material according to claim 5, wherein the pulp is hardwood pulp.

9. The molding material according to claim 5, wherein 0.3 to 2.0 parts by mass of a non-alkali metal salt of long-chain fatty acid has been added to 100 parts by mass of a mixture of the pulp, the carboxymethyl cellulose salt, and the starch.

10. A joining structure formed by joining pieces by engaging and pressurizing the pieces with the pieces moistened, and solidifying the pieces by drying, wherein the pieces are formed by molding the molding material according to claim 5, and not for an applicator for intravaginal insertion.

11. The joining structure according to claim 10, wherein the joining by engaging and pressurizing is performed by applying 10 g/m² or more of water to an engaged portion and retaining an applied pressure equal to or higher than 10 kPa for 10 sec or longer.

12. A method for producing a molding material comprising pulp, carboxymethyl cellulose salt, starch, and water for use in injection molding or compression molding, wherein the pulp, the carboxymethyl cellulose salt, the starch, and the water are mixed and kneaded.

13-16. (canceled)

17. The method for producing a molding material according to claim 12, wherein a product obtained by mixing the pulp, the carboxymethyl cellulose salt, the starch, and the water is kneaded with addition of a non-alkali metal salt of long-chain fatty acid.

18. (canceled)

19. The method for producing a molding material according to claim 12, wherein the kneading is performed at 40° C. to 90° C.

20. A method for producing a molded article comprising pulp, carboxymethyl cellulose salt, and starch, wherein injection molding or compression molding is performed with use of the molding material according to claim 5 under the following conditions:
injection speed: 10 to 30 mm/s;
injection pressure: 80 to 140 MPa; and
mold temperature: 150 to 180° C.

21. A method for joining molded articles according to claim 1, the method comprising:
an application step of applying water to facing surfaces for joining the molded articles together;
an engaging step of engaging the facing surfaces with each other; and
a drying step of drying a resulting engaged portion.

22-25. (canceled)

26. The method for joining molded articles according to claim 21, wherein the application step is performed under the following condition:
water application rate: 10 g/m² or more.

27. The method for joining molded articles according to claim 21, wherein the engaging step is performed under the following conditions:
applied pressure: 10 kPa or higher; and
pressurization retention time: 10 sec or longer.

28. The method for joining molded articles according to claim 21, wherein the drying step is performed under the following conditions:
temperature: 50° C. or higher; and
drying time: 2 minutes or longer.
